(19) United States
(12) Patent Application Publication
(10) Pub. No.: US 2020/0308646 A1
(43) Pub. Date: Oct. 1, 2020

(54) METHODS FOR DIAGNOSING INFLAMMATORY PHENOTYPES OF CHRONIC OBSTRUCTIVE PULMONARY DISEASE

(71) Applicants: The University of Newcastle, Callaghan, New South Wales (AU); Hunter New England Local Health District, New Lambton, New South Wales (AU)

(72) Inventors: Peter G. Gibson, Merewether (AU); Katherine J. Baines, Floraville (AU)

(21) Appl. No.: 16/624,955

(22) PCT Filed: Jun. 26, 2018
(86) PCT No.: PCT/AU2018/050644

§ 371 Date: Dec. 20, 2019

(30) Foreign Application Priority Data
Jun. 26, 2017 (AU) 2017902450

(51) Int. Cl. C12Q 1/6883

(52) U.S. Cl. CPC C12Q 1/6883; C12Q 2600/112; G01N 2800/122; C12Q 2600/158; G01N 2800/52; C12Q 2600/118

(57) ABSTRACT

Provided herein are methods for diagnosing the COPD inflammatory phenotype of an individual, and methods for selecting individuals with COPD for treatment based on their COPD inflammatory phenotype. In particular embodiments the methods comprise determining the level of expression of one or more genes selected from CLC, CPA3, DNASE1L3, IL1B, ALPL and CXCR2, or determining the expression profile CLC, CPA3, DNASE1L3, IL1B, ALPL and CXCR2, in one or more biological samples from an individual, wherein the expression levels or expression profile are indicative of the COPD inflammatory phenotype.

### CROSS-REFERENCE TO RELATED APPLICATIONS

This application is a U.S. national stage filing, under 35 U.S.C. § 371(c), of International Application No. PCT/AU2018/050644, filed on Jun. 26, 2018, which claims priority to Australian Patent Application No. 2017902450, filed on Jun. 26, 2017.

### FIELD OF THE ART

The present disclosure relates generally to predicting a subject's inflammatory phenotype of chronic pulmonary disease (COPD) based on the differential expression of a panel of biomarkers.

### BACKGROUND

Chronic obstructive pulmonary disease (COPD) is a common inflammatory airway disease and is a major cause of chronic morbidity. Individuals with COPD may present with chronic cough, sputum production, dyspnea and there may be permanent and/or progressive airway obstruction and damage to alveoli. Exacerbations of COPD are defined as periods of acute deterioration of symptoms and lung function that can result in hospitalization and increased health care utilization. Exacerbations impose a substantial economic burden and result in a faster decline in lung function and poorer quality of life and are a major cause of death. Some patients experience frequent exacerbations that require more effective management strategies. A full understanding of pathogenesis of COPD and recognition of disease heterogeneity is crucial for improvement of COPD management and treatment.

Inflammation in COPD has both airway and systemic components. Low grade systemic inflammation is associated with a rapid decline in lung function, increased mortality, and a higher exacerbation rate. Indeed, the inventors have recently shown that the presence of systemic inflammation, measured by elevated systemic C-reactive protein (CRP) and interleukin (IL)-6, is predictive of future exacerbations in COPD (Fu et al., 2015, Chest, 148:618-629). Systemic inflammation was also associated with elevated interleukin (IL)-1β expression in the airways, and this airway-systemic inflammatory axis was predictive of COPD exacerbations.

Neutrophilic airway inflammation is often associated with COPD and increased neutrophils in sputum is thought to correlate with peripheral airway dysfunction in smokers. More recently, eosinophilic airway inflammation has been identified in a subset of patients with COPD, although this symptom was classically thought to be characteristic of asthma. It therefore appears that COPD is a heterogeneous inflammatory disorder and different individuals may be characterised by different types of inflammation, possibly indicating different subtypes of COPD or different stages of disease severity or progression.

Lifestyle changes, such as cessation of smoking and avoiding airway irritants, are often advised in the management of COPD. Oxygen therapy or surgery may also be used to treat some patients with COPD or bronchodilators may be used to relax muscles of the airways to reduce symptoms. Generally, for more severe COPD, inhaled glucocorticosteroids may be prescribed to reduce airway inflammation. Typically, few of the therapy options are effective, although COPD patients with eosinophilic inflammation tend to be more responsive to corticosteroid treatment than other COPD patients. Determining a patient's inflammatory phenotype would therefore be of significant assistance in identifying the most effective treatment. The development of molecular signatures are likely to aid a personalised phenotype based airways disease management and treatment approaches.

### SUMMARY OF THE DISCLOSURE

An indication of the COPD inflammatory phenotype of an individual can be obtained by assessing inflammatory cells at the site of inflammation. These cells may be quantified in a sputum sample from the patient. An alternative, less labour intensive method is to measure expression of genes in the sputum sample. The present disclosure describes, inter alia, a six gene signature that can be measured in an individual with COPD to determine the COPD inflammatory phenotype of the individual, which signature therefore also informs the appropriate treatment for the COPD inflammatory phenotype suffered by the individual.

Accordingly, provided herein are methods for distinguishing between COPD inflammatory phenotypes in a subject suffering from COPD, comprising determining the level of expression of one or more genes selected from CLC, CPA3, DNASE1L3, IL1B, ALPL and CXCR2 in a biological sample isolated from the subject, wherein the expression profile of the one or more genes in the sample is indicative of the COPD inflammatory phenotype in the subject, and wherein the COPD inflammatory phenotype is selected from eosinophilic COPD, neutrophilic COPD, paucigranulocytic COPD and mixed granulocytic COPD. Also provided herein are methods of treatment for the COPD inflammatory phenotype suffered by a subject, wherein the method of treatment employed is predicated on the determination or measurement of the level of expression of one or more genes selected from CLC, CPA3, DNASE1L3, IL1B, ALPL and CXCR2 in a biological sample isolated from the subject, wherein the expression profile of the one or more genes in the sample is indicative of the COPD inflammatory phenotype in the subject, and wherein the COPD inflammatory phenotype is selected from eosinophilic COPD, neutrophilic COPD, paucigranulocytic COPD and mixed granulocytic COPD.

A first aspect of the present disclosure provides a method for determining an inflammatory phenotype of chronic obstructive pulmonary disease (COPD) in a subject with COPD, the method comprising:
determining the level of expression of one or more genes in a biological sample from the subject, wherein the one or more genes are selected from CLC, CPA3, DNASE1L3, IL1B, ALPL and CXCR2;
   wherein the level of expression of the one or more genes is indicative of the COPD inflammatory phenotype of the subject.

In an embodiment, the method comprises obtaining the biological sample from the subject.

In an embodiment the COPD inflammatory phenotype is selected from eosinophilic COPD, neutrophilic COPD, paucigranulocytic COPD and mixed granulocytic COPD.

Expression of the one or more genes may be determined at the mRNA or gene level, or at the polypeptide or protein level. Typically, the correlation between expression of the one or more genes and a COPD inflammatory phenotype is determined by statistical analysis of the mRNA or protein expression levels, such as by logistic regression analysis.

To determine the COPD inflammatory phenotype, the level of expression of the one or more genes may be compared to the level of expression of the same gene(s) in one or more reference samples. The one or more reference samples may be from one or more individuals known to have COPD. The COPD inflammatory phenotype in said one or more individuals may be known. Alternatively, the one or more reference samples may be from one or more individuals known not to have COPD.

In an embodiment, elevated expression of one or more of CLC, CPA3 and/or DNASE1L3 in the biological sample, compared to one or more reference samples from one or more individuals known to have COPD, is indicative of eosinophilic COPD. Typically the one or more reference samples are from one or more individuals known not to have eosinophilic COPD.

In an embodiment, elevated expression of one or more of IL1B, ALPL and/or CXCR2 in the biological sample, compared to one or more reference samples from one or more individuals known to have COPD, is indicative of neutrophilic COPD. Typically the one or more reference samples are from one or more individuals known not to have neutrophilic COPD.

In an embodiment, elevated expression of IL1B in the biological sample, compared to one or more reference samples from one or more individuals known to have COPD, is indicative of non-eosinophilic COPD. Typically the one or more reference samples are from one or more individuals known to have eosinophilic COPD.

In an embodiment, the combined expression profile of CLC, CPA3, DNASE1L3, IL1B, ALPL and CXCR2 in the biological sample is compared to the combined expression profile of said genes in one or more reference samples from one or more individuals known to have COPD. The COPD inflammatory phenotype of said one or more individuals may be known.

In a particular embodiment, the biological sample is sputum. The sputum may be induced sputum. The present disclosure provides that sputum may be induced in a subject using methods known to those in the art, for example with the use of hypertonic saline (4.5%) if the subject's forced expiratory volume in 1 second (FEV₁) is more than or equal to 1L. In further exemplary embodiments, if the subject's FEV₁ is less than 1L, 0.9% saline may be used to induce sputum. In an embodiment, inflammatory cells, preferably non-squamous cells, may also be quantified in the biological sample.

In an embodiment of the first aspect the subject is administered a treatment for the inflammatory phenotype of COPD determined on the basis of the level of expression of the one or more genes.

Accordingly, a second aspect of the present disclosure provides a method for treating an inflammatory phenotype of COPD, the method comprising:
i) determining the level of expression of one or more genes in a biological sample from the subject, wherein the one or more genes are selected from CLC, CPA3, DNASE1L3, IL1B, ALPL and CXCR2, and wherein the level of expression of the one or more genes is indicative of the COPD inflammatory phenotype of the subject; and
ii) treating the subject for the COPD inflammatory phenotype indicated in i).

A third aspect of the present disclosure provides a method for determining a COPD inflammatory phenotype in a subject with COPD, the method comprising: determining the expression profile, in a biological sample from the subject, of the genes CLC, CPA3, DNASE1L3, IL1B, ALPL and CXCR2; wherein the expression profile of said genes is indicative of the COPD inflammatory phenotype of the subject.

In an embodiment the method comprises obtaining the biological sample from the subject.

Typically, the correlation between the expression profile of the genes and a COPD inflammatory phenotype is determined by statistical analysis of the mRNA or protein expression levels, such as by logistic regression analysis.

To determine the COPD inflammatory phenotype, the level of expression of the one or more genes may be compared to the level of expression of the same gene(s) in one or more reference samples. The one or more reference samples may be from one or more individuals known to have COPD. The COPD inflammatory phenotype in said one or more individuals may be known. Alternatively, the one or more reference samples may be from one or more individuals known not to have COPD.

In an embodiment of the third aspect the method enables the determination of whether the subject has eosinophilic COPD or non-eosinophilic COPD. Said determination may be based on multiple logistic regression analysis of the expression profile or expression levels.

In an embodiment of the third aspect multiple logistic regression analysis of the expression profile or expression levels of said genes enables the distinction between:
a) eosinophilic COPD and non-eosinophilic COPD;
b) eosinophilic COPD and neutrophilic COPD;
c) eosinophilic COPD and paucigranulocytic COPD;
d) eosinophilic COPD and mixed granulocytic COPD;
e) neutrophilic COPD and non-neutrophilic COPD;
f) neutrophilic COPD and paucigranulocytic COPD;
g) neutrophilic COPD and mixed granulocytic COPD;
h) paucigranulocytic COPD and non-paucigranulocytic COPD; or
i) paucigranulocytic COPD and mixed granulocytic COPD.

In an embodiment of the third aspect the subject is administered a treatment for the inflammatory phenotype of COPD determined on the basis of the level of expression of the one or more genes.

Accordingly, a fourth aspect of the present disclosure provides a method for treating an inflammatory phenotype of COPD, the method comprising:
i) determining the expression profile of the genes CLC, CPA3, DNASE1L3, IL1B, ALPL and CXCR2 in a biological sample from the subject, wherein the expression profile is indicative of the COPD inflammatory phenotype of the subject; and
ii) treating the subject for the COPD inflammatory phenotype indicated in i).

A fifth aspect of the present disclosure provides a method for selecting a subject for treatment of an inflammatory phenotype of COPD, comprising:
i) executing the step of determining the level of expression of one or more genes in a biological sample from a subject, wherein the one or more genes are selected from CLC, CPA3, DNASE1L3, IL1B, ALPL and CXCR2;
ii) determining the COPD inflammatory phenotype based on the determination in i); and
iii) selecting a subject for treatment for said COPD inflammatory phenotype determined in ii).

A sixth aspect of the present disclosure provides a method for selecting a subject for treatment of an inflammatory phenotype of COPD, comprising:
i) executing the step of determining the expression profile, in a biological sample from a subject, of the genes CLC, CPA3, DNASE1L3, IL1B, ALPL and CXCR2;
ii) determining the COPD inflammatory phenotype based on the determination in i); and
iii) selecting a subject for treatment for said COPD inflammatory phenotype determined in ii).

In a further aspect of the present disclosure, a method is provided for determining a treatment regime for a subject suffering from COPD, the method comprising determining the COPD inflammatory phenotype in the subject in accordance with the first or second aspect and selecting an appropriate treatment regime for the subject on the basis of the determination.

In embodiments of the above aspect the treatment regime may comprise treatment with bronchodilators or corticosteroids.

### BRIEF DESCRIPTION OF THE DRAWINGS

Aspects and embodiments of the present disclosure are described herein, by way of non-limiting example only, with reference to the following drawings.

FIG. 1: Relative gene expression levels of A) CLC, B) CPA3, C) DNASE1L3, D) IL1B, E) ALPL and F) CXCR2 in induced sputum samples from subjects with eosinophilic (E), neutrophilic (N), paucigranulocytic (PG) or mixed granulocytic (MG) COPD. Gene expression is calculated relative to β-actin (ΔCt), log transformed (2^{-ΔCt}) and scaled. Bar graphs show the median and error bars as the 95% CI. ** p<0.01 versus PG-COPD; #p<0.01 versus N-COPD; *p<0.05 versus PG-COPD; ∧p<0.01 versus E-COPD; ˜p<0.05 versus E-COPD.

FIG. 2: Receiver operating characteristic (ROC) curves demonstrate that the sputum 6 gene expression biomarker signature discriminates A) eosinophilic COPD from non-eosinophilic COPD, and B) neutrophilic from non-neutrophilic COPD.

FIG. 3: Receiver operating characteristic curves demonstrates that the 6 gene expression biomarker signature discriminates A) E-COPD from N, PG and MG-COPD, B) N-COPD from PG and MG-COPD and C) MG-COPD from PG-COPD.

FIG. 4: Sputum gene expression of IL1B correlates with A) FEV1% predicted; B) FEV1/FVC; C) Charlson Comorbidity Index (CCI); D) C Reactive Protein (hs-CRP); E) GOLD stage and F) BODE index. **p<0.001 versus GOLD stage 1 and GOLD stage 2; #p<0.05 versus GOLD stage 3.

FIG. 5: Bland-Altman plots for the E-COPD markers CLC (A), CPA3 (B) and DNASE1L3 (C), and the N-COPD markers IL1B (D), ALPL (E) and CXCR2 (F). Bland-Altman plots indicate the mean ΔCt against the absolute difference in ΔCt between visits for each gene. Horizontal dotted lines represent the 95% limits of agreement (mean difference ±1.96 SD).

The subject specification contains amino acid and nucleotide sequence information prepared using the programme Patentln Version 3.5, presented herein in a Sequence Listing. The instant application contains a Sequence Listing which has been submitted electronically in ASCII format and is hereby incorporated by reference in its entirety. Said ASCII copy, created on Mar. 10, 2020, is named 119134-02501_SL.txt and is 26, 253 bytes in size. The nucleotide sequences of the human CLC, CPA3, DNASE1L3, IL1B, ALPL, and CXCR2 genes are provided in SEQ ID NOs:1, 3, 5, 7, 9, and 11, respectively. The amino acid sequences of the polypeptides encoded by these genes are provided in SEQ ID NOs:2, 4, 6, 8, 10, and 12, respectively.

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

“CLC” refers to the gene encoding the Charcot-Leyden crystal protein. Whilst the present disclosure typically refers to the gene and polypeptide as found in humans, or to derivatives, fragments or variants thereof, those skilled in the art will appreciate that homologues of human from other species are also contemplated and encompassed. The cDNA encoding human CLC is located in the National Center for Biotechnology Information (NCBI) database as Accession No. NM_001828.5. An exemplary nucleotide sequence of human CLC is set forth in SEQ ID NO:1, and an exemplary encoded polypeptide sequence is set forth in SEQ ID NO:2.

“CPA3” refers to the gene encoding carboxypeptidase A3. Whilst the present disclosure typically refers to the gene and polypeptide as found in humans, or to derivatives, fragments or variants thereof, those skilled in the art will appreciate that homologues of human from other species are also contemplated and encompassed. The cDNA encoding human CPA3 is located in the National Center for Biotechnology Information (NCBI) database as Accession No. NM_001870.2. An exemplary nucleotide sequence of human CPA3 is set forth in SEQ ID NO:3, and an exemplary encoded polypeptide sequence is set forth in SEQ ID NO:4.

“DNASE1L3” refers to the gene encoding deoxyribonuclease I-like 3. Whilst the present disclosure typically refers to the gene and polypeptide as found in humans, or to derivatives, fragments or variants thereof, those skilled in the art will appreciate that homologues of human from other species are also contemplated and encompassed. The cDNA encoding human DNASE1L3 is located in the National Center for Biotechnology Information (NCBI) database as Accession No. NM_004944.3. An exemplary nucleotide sequence of human DNASE1L3 is set forth in SEQ ID NO:5, and an exemplary encoded polypeptide sequence is set forth in SEQ ID NO:6.

“IL1B” refers to the gene encoding interleukin-1β. Whilst the present disclosure typically refers to the gene and polypeptide as found in humans, or to derivatives, fragments or variants thereof, those skilled in the art will appreciate that homologues of human from other species are also contemplated and encompassed. The cDNA encoding human IL1B is located in the National Center for Biotechnology Information (NCBI) database as Accession No. NM_000576.2. An exemplary nucleotide sequence of human IL1B is set forth in SEQ ID NO:7, and an exemplary encoded polypeptide sequence is set forth in SEQ ID NO:8.

“ALPL” refers to the gene encoding alkaline phosphatase, tissue-nonspecific isozyme. Whilst the present disclosure typically refers to the gene and polypeptide as found in humans, or to derivatives, fragments or variants thereof, those skilled in the art will appreciate that homologues of human from other species are also contemplated and encompassed. The cDNA encoding human ALPL is located in the National Center for Biotechnology Information (NCBI) database as Accession No. NM_000478.4. An exemplary nucleotide sequence of human ALPL is set forth in SEQ ID NO:9, and an exemplary encoded polypeptide sequence is set forth in SEQ ID NO:10.

“CXCR2” refers to the gene encoding chemokine (C-X-C motif) receptor 2, also known as IL8RB (interleukin-8 receptor B). Reference herein to CXCR2 should be understood to be a reference to IL8RB. Whilst the present disclosure typically refers to the gene and polypeptide as found in humans, or to derivatives, fragments or variants thereof, those skilled in the art will appreciate that homologues of human from other species are also contemplated and encompassed. The cDNA encoding human CXCR2 is located in the National Center for Biotechnology Information (NCBI) database as Accession No. NM_001557.3. An exemplary nucleotide sequence of human CXCR2 is set forth in SEQ ID NO:11, and an exemplary encoded polypeptide sequence is set forth in SEQ ID NO:12.

As used herein the term “gene” means a nucleic acid molecule having a particular function. As such the term “gene” encompasses not only the genomic nucleic acid molecule, but also the mRNA product of the genomic molecule, and equivalent cDNA molecules, as well as functionally equivalent genomic variants, derivatives, alternative splicing variants and genetic isoforms of the gene. Variants and derivatives typically exhibit at least some of the functional activity of the gene of which it is a variant or derivative.

As used herein the term “protein” means a peptide or polypeptide molecule having a particular function. As such the term “protein” encompasses not only the peptide or polypeptide product of a gene, but also functionally equivalent fragments, derivatives and variants thereof and post-translationally modified forms of the peptide or polypeptide product. Variants and derivatives typically exhibit at least some of the functional activity of the gene of which it is a variant or derivative. Different isoforms of a protein are also encompassed by this general term. Also encompassed by the term “protein” as used herein are mature protein and polypeptide sequences, in addition to proproteins, preproproteins and other precursor molecules including, for example, signal peptides, activation peptides or other sequences cleaved from a precursor molecule to generate the mature protein or polypeptide sequence.

As used herein the term “expression profile” may refer to the expression level of one or more genes or proteins in a given sample or to a value determined from the expression level of one or more genes or proteins. Such a value may be determined by statistical analysis of expression levels as described herein. The expression profile of a group or set of two or more genes or proteins may be referred to herein as a ‘combined expression profile’. Expression levels of genes and proteins may be measured for example by any suitable method for determining, and typically quantifying, gene and protein expression known to those skilled in the art. The person of skill in the art can determine the most appropriate means of analysis in any given situation.

In the context of the present disclosure, reference to an increase or decrease in an expression profile or combined expression profile in a given sample means an increase or decrease in the level of expression of the gene(s) or protein(s) in question in the sample, typically when compared to the expression levels in one or more reference or control samples. In some embodiments an increase or decrease in an expression profile or combined expression profile in a given sample obtained from a subject following therapy or treatment may refer to an increase or decrease in the level of expression of the gene(s) or protein(s) in question when compared to the expression levels prior to, or in the absence of, said therapy or treatment.

The expression profile employed in methods disclosed herein may be subject to, or may result from statistical analysis of expression levels, for example, in comparing expression profiles between samples including reference or control samples. Statistical techniques that may be employed for such analyses are known to those skilled in the art and include, but are not limited to, meta-analysis, multiple regression analysis, and receiver operator curve (ROC) analysis. ROC analysis is used to determine a score that is diagnostic with the greatest sensitivity and specificity. The ‘squarer’ the look of the curve, the simpler the determination of a diagnostic level or score. The closer the area under the curve is to 1 also indicates a result with high sensitivity and specificity.

In the context of this specification, the term “expression signature” is used to describe the expression profile of a combination of two or more biomarker genes from the same subject. Typically, the two or more biomarkers will be measured in the same sample. As used herein, the term “6-gene signature” is used to describe the combined expression profiles of CLC, CPA3, DNASE1L3, IL1B, ALPL and CXCR2. The expression profile for a biomarker typically comprises the expression level of the RNA. RNA may be measured for example by quantifying RNA expression, methods of which are well known to those skilled in the art. Alternatively expression may be measured at the level of protein, also using techniques and methods known to those skilled in the art. The person of skill in the art will determine the most appropriate means of analysis in any given situation.

In the context of this specification, the term “phenotype” is used to refer to a physical or physiological character or any observable or implied characteristic, state or trait of an inflammatory condition.

As used herein, the term “subject” may be used interchangeably with the term “individual” or “participant”. A “subject” may include any mammal, such as humans, non-human primates, livestock animals (eg. sheep, pigs, cattle, horses, donkeys, goats), laboratory test animals (eg. mice, rabbits, rats, guinea pigs, other rodents), companion animals (eg. dogs, cats). In a preferred embodiment, the subject is a human.

As used herein the term “treatment” refers to any and all treatments that remedy a condition or one or more symptoms of a condition or disease, prevent the establishment of a condition or disease, or otherwise prevent, hinder, retard, or reverse the progression of a condition or disease or other undesirable symptoms in any way whatsoever. Thus the term “treatment” is to be considered in its broadest context. For example, treatment does not necessarily imply that a patient is treated until total recovery.

Effective clinical management of COPD requires objective measurement of inflammatory phenotype. However, the most direct measures of airway inflammation are too invasive and have limited clinical use. By using the gene expression analysis described' herein the present inventors have identified that the combined expression profile of specific genes can distinguish between inflammatory phenotypes of COPD, and thus can also be employed to predict and monitor patient response to therapeutic intervention.

Specifically, as exemplified herein, the inventors have identified genes capable of serving as non-invasive discriminatory biomarkers, based on expression levels determined from sputum samples. The genes are selected from CLC, CPA3, DNASE1L3, IL1B, ALPL, and CXCR2. In particular the expression signature or profile of these six genes is able to distinguish between COPD inflammatory phenotypes. The study described herein has shown that the 6 gene expression signature of CLC, CPA3, DNASE1L3, IL1B, ALPL and CXCR2 can distinguish between patients with different inflammatory phenotypes of COPD, with a substantial degree of accuracy and reproducibility. In agreement with the inventors' previous findings in asthma, CLC, CPA3 and DNASE1L3 were found to have increased gene expression in patients with eosinophilic airway inflammation, whereas expression levels of IL1B, ALPL and CXCR2 were high in those with neutrophilic airway inflammation. Elevated expression levels of genes associated with neutrophilic inflammation, in particular IL1B, were associated with poorer lung function, systemic inflammation, comorbidity, severity and a higher BODE index.

Evidence in the literature suggests that treatment with either OCS or ICS have little effect in lessening neutrophilic airway inflammation in COPD, and new treatment approaches including selective phosphodiesterase (PDE) inhibitors and macrolide antibiotics are thus being tested. As has been recognised in the art, the key to the success of clinical trials on novel treatment approaches targeting airway inflammation in COPD mainly depends on the ability to accurately phenotype patients, which can provide information as to possible mechanisms, mediators or cytokines involved in the disease pathogenesis. In relation to this, the development described herein of a sputum 6-gene expression signature capable of distinguishing inflammatory phenotypes of COPD, and in particular the ability of this signature to discriminate neutrophilic COPD from non-neutrophilic COPD is highly significant as it may enable identifying novel treatment targets.

Provided herein are methods for determining the inflammatory phenotype (or distinguishing between inflammatory phenotypes) in COPD sufferers.

One aspect of the present disclosure provides a method for determining an inflammatory phenotype of chronic obstructive pulmonary disease (COPD) in a subject with COPD, the method comprising: determining the level of expression of one or more genes in a biological sample from the subject, wherein the one or more genes are selected from CLC, CPA3, DNASE1L3, IL1B, ALPL and CXCR2;

wherein the level of expression of the one or more genes is indicative of the COPD inflammatory phenotype of the subject.

The expression the biomarker genes may be measured alone or in various combinations to distinguish inflammatory phenotypes of an individual with COPD. Exemplary embodiments measure the expression level of two or more, three or more, four or more, five or more or all of CLC, CPA3, DNASE1L3, IL1B, ALPL and CXCR2.

In an embodiment, elevated expression of one or more of CLC, CPA3 and/or DNASE1L3 in the biological sample, compared to one or more reference samples from one or more individuals known to have COPD, is indicative of eosinophilic COPD. Typically the one or more reference samples are from one or more individuals known not to have eosinophilic COPD.

In an embodiment, elevated expression of one or more of IL1B, ALPL and/or CXCR2 in the biological sample, compared to one or more reference samples from one or more individuals known to have COPD, is indicative of neutrophilic COPD. Typically the one or more reference samples are from one or more individuals known not to have neutrophilic COPD.

In an embodiment, elevated expression of IL1B in the biological sample, compared to one or more reference samples from one or more individuals known to have COPD, is indicative of non-eosinophilic COPD. Typically the one or more reference samples are from one or more individuals known to have eosinophilic COPD.

In an embodiment, the combined expression profile of CLC, CPA3, DNASE1L3, IL1B, ALPL and CXCR2 in the biological sample is compared to the combined expression profile of said genes in one or more reference samples from one or more individuals known to have COPD. The COPD inflammatory phenotype of said one or more individuals may be known.

Another aspect of the present disclosure provides a method for determining a COPD inflammatory phenotype in a subject with COPD, the method comprising:
determining the expression profile, in a biological sample from the subject, of the genes CLC, CPA3, DNASE1L3, IL1B, ALPL and CXCR2;
   wherein the expression profile of said genes is indicative of the COPD inflammatory phenotype of the subject.

Methods disclosed herein enable the distinction between:
(i) eosinophilic COPD and non-eosinophilic COPD;
(ii) eosinophilic COPD and neutrophilic COPD;
(iii) eosinophilic COPD and paucigranulocytic COPD;
(iv) eosinophilic COPD and mixed granulocytic COPD;
(v) neutrophilic COPD and non-neutrophilic COPD;
(vi) neutrophilic COPD and paucigranulocytic COPD;
(vii) neutrophilic COPD and mixed granulocytic COPD;
(viii) paucigranulocytic COPD and non-paucigranulocytic COPD; or
(ix) paucigranulocytic COPD and mixed granulocytic COPD.

Typically, the correlation between expression of the gene(s) or protein(s) and a COPD inflammatory phenotype is determined by statistical analysis of the expression levels or expression profile, such as by logistic regression analysis, as described herein.

The biological sample obtained from a subject in accordance with the present disclosure may be any suitable biological sample. The term “biological sample” is used to refer to any material, biological fluid, tissue, or cell obtained from a subject, including but not limited to blood, sputum, mucus, saliva, bronchial aspirate, cells and cellular extracts. The biological sample may be obtained by any suitable method, which may be determined by a person skilled in the art. In particular embodiments, the biological sample is sputum. The sputum may be induced sputum. The sputum may be induced in a subject using methods known to those in the art, for example with the use of hypertonic saline (4.5%) if the subject's forced expiratory volume in 1 second (FEV₁) is more than or equal to 1L. In further exemplary embodiments, if the subject's FEV₁ is less than 1L, 0.9% saline may be used to induce sputum.

A subject identified, in accordance with the methods of the present disclosure described hereinbefore as having a COPD inflammatory phenotype, can be selected for treatment, or stratified into a treatment group, wherein an appropriate therapeutic regimen can be adopted or prescribed with a view to treating the condition.

Thus, in an embodiment, the methods disclosed herein may comprise the step of exposing (i.e., subjecting) a subject identified as having a COPD inflammatory phenotype to a therapeutic treatment or regimen for treating the condition. The nature of the therapeutic treatment or regimen to be employed can be determined by persons skilled in the art and will typically depend on factors such as, but not limited to, the age, weight and general health of the subject.

An aspect of the present disclosure therefore provides a method for selecting a subject for treatment of an inflammatory phenotype of COPD, comprising:
i) executing the step of determining the level of expression of one or more genes in a biological sample from a subject, wherein the one or more genes are selected from CLC, CPA3, DNASE1L3, IL1B, ALPL and CXCR2;
ii) determining the COPD inflammatory phenotype based on the determination in i); and
iii) selecting a subject for treatment for said COPD inflammatory phenotype determined in ii).

Another aspect of the present disclosure provides a method for selecting a subject for treatment of an inflammatory phenotype of COPD, comprising:
i) executing the step of determining, in a biological sample from a subject, the expression profile of the genes CLC, CPA3, DNASE1L3, IL1B, ALPL and CXCR2;
ii) determining the COPD inflammatory phenotype based on the determination in i); and
iii) selecting a subject for treatment for said COPD inflammatory phenotype determined in ii).

A further aspect provides a method for treating an inflammatory phenotype of COPD, the method comprising:
i) determining the level of expression of one or more genes in a biological sample from the subject, wherein the one or more genes are selected from CLC, CPA3, DNASE1L3, IL1B, ALPL and CXCR2, and wherein the level of expression of the one or more genes is indicative of the COPD inflammatory phenotype of the subject; and
ii) treating the subject for the COPD inflammatory phenotype indicated in i).

A further aspect provides a method for treating an inflammatory phenotype of COPD, the method comprising:
i) determining the expression profile of the CLC, CPA3, DNASE1L3, IL1B, ALPL and CXCR2 in the biological sample, wherein the expression profile is indicative of the COPD inflammatory phenotype of the subject; and
ii) treating the subject for the COPD inflammatory phenotype indicated in i).

It will be clear to the skilled addressee that the methods disclosed herein can be also used to monitor the response of a subject to, and the efficacy of, treatment of COPD, whereby the level of expression of one or more genes selected from CLC, CPA3, DNASE1L3, IL1B, ALPL and CXCR2, or the expression profile of the CLC, CPA3, DNASE1L3, IL1B, ALPL and CXCR2 genes, may be determined at two or more separate time points, optionally including before commencement of treatment, during the course of treatment and after cessation of treatment, to determine whether said treatment is effective.

Thus, the disclosure provides a method for monitoring the response of a subject to a therapeutic treatment for COPD, the method comprising:
i) obtaining from a subject a first biological sample before or after commencement of therapeutic treatment;
ii) executing the step of determining the level of expression of one or more genes selected from CLC, CPA3, DNASE1L3, IL1B, ALPL and CXCR2, or the expression profile of the CLC, CPA3, DNASE1L3, IL1B, ALPL and CXCR2 genes in the first biological sample;
iii) obtaining from the same subject a second biological sample at a time point after commencement of treatment and after the first biological sample is obtained;
iv) executing the step of determining the level of expression of one or more genes selected from CLC, CPA3, DNASE1L3, IL1B, ALPL and CXCR2, or the expression profile of the CLC, CPA3, DNASE1L3, IL1B, ALPL and CXCR2 genes in the second biological sample; and
v) comparing said levels of expression, or expression profiles, from the first and second samples,
   wherein the comparison is indicative of whether or not the subject is responding to the therapeutic treatment.

The above described method may further comprise obtaining and executing steps in respect of a third or subsequent sample. A change in said expression levels or expression profiles from the first and second (or subsequent) sample may be indicative of an effective therapeutic treatment or regimen and positive response of the subject to a treatment. Where the method or protocol indicates that the therapeutic treatment or regimen is ineffective and/or the subject is not responding sufficiently to the treatment (i.e. no or insignificant change in expression levels or expression profiles), the method or protocol may further comprise altering or otherwise modifying the therapeutic treatment or regimen with a view to providing a more efficacious or aggressive treatment. This may comprise administering to the subject additional doses of the same agent with which they are being treated or changing the dose and/or type of medication or other treatment.

It should be understood that reference herein to determining the level of expression of a gene is intended as a reference to the use of any suitable technique that will provide information in relation to the level of expression of the encoding nucleic acid molecule (DNA or mRNA) or the encoded protein or polypeptide in the relevant tissue of the subject. Accordingly, these techniques include both in vivo techniques, as well as in vitro techniques that are applied to a biological sample extracted from the subject. Such in vitro techniques are likely to be preferred due to their significantly more simplistic and routine nature. Those skilled in the art will readily appreciate that in accordance with embodiments of the present disclosure gene expression may be determined by any suitable technique or assay known in the art. Methods disclosed herein typically require quantitation of expression levels. Analysis of gene expression at the level of the mRNA may use amplification based assays such as reverse-transcription PCR (RT-PCR) coupled with real time quantitative PCR (qPCR). Other suitable methods include but are not limited to microarrays, ligase chain reaction, oligonucleotide ligation assay, next generation sequencing, northern blotting, in situ hybridisation and further statistical analysis to determine differential expression. Exemplary methods for determining expression at the protein or polypeptide level include, for example, immunoassay using an antibody(ies) that bind with the protein such as enzyme-linked immunosorbent assay (ELISA) or immunoblotting, 2D-gel electrophoresis (including 2D-DIGE), multiplex protein expression assays, western blotting, immunoprecipitation assays, HPLC, LC/MS, flow cytometry and protein expression profiling arrays and microarrays. The skilled addressee will appreciate that the present disclosure is not limited by reference to the means by which gene expression is determined and/or quantified.

In embodiments of the present disclosure, gene expression may be measured in conjunction with quantification of one or more other markers of inflammation, such as inflammatory cells. Inflammatory cells may be identified and quantified in the biological samples by methods known to those in the art. Inflammatory cell counts, for example of non-squamous cells, may be performed using methods known in the art. Preparing samples for quantification of inflammatory cells may comprise dispersing sputum using a reducing agent, such as dithiothreitol and preparing cytospins. In embodiments, the cells may be stained, such as using a May-Grunwald—Giemsa stain, hematoxylin and eosin stain, toluidine staining or immunostaining.

Methods of the present disclosure may be employed to determine or distinguish between COPD inflammatory phenotypes either in subjects that are known to have COPD (symptomatic or asymptomatic) or in subjects suspected of having COPD. Moreover, embodiments of the present disclosure may be used alone or in conjunction with, or as an adjunct to, one or more other diagnostic methods and tests to determine COPD per se, or the COPD inflammatory phenotype experienced by a subject. Such other diagnostic methods and tests will be well known to those skilled in the art.

Thus, COPD may be diagnosed in a subject by any method available in the art. Suitable methods are well known to those in the art, such as spirometry. For example, COPD may be confirmed by incompletely reversible airflow limitation, that is a post-bronchodilator forced expiratory volume in 1 second (FEV₁) of <80% predicted and FEV₁ to forced vital capacity (FVC) ratio of <0.70. As exemplified herein inflammatory phenotypes of COPD may be characterised as follows: eosinophilic COPD, wherein the sputum eosinophil count is more than or equal to about 3%; neutrophilic COPD, wherein the sputum neutrophil count is more than or equal to about 61%; paucigranulocytic COPD, wherein the sputum eosinophil count is less than about 3% and the sputum neutrophil count is less than about 61%; and mixed granulocytic COPD, wherein the sputum eosinophil count is more than or equal to about 3% and the sputum neutrophil count is more than about 61%. Those skilled in the art will appreciate that these criteria for different COPD inflammatory phenotypes is exemplary only, and the cut-off values (or indeed the assessment criteria) may be varied based on the skilled addressees understanding of COPD and the inflammation associated with COPD.

The expression levels or profiles determined in samples from subjects in accordance with methods of the present disclosure can be compared to reference or control values as a suitable reference to assist in diagnosis, for example such that abnormal expression levels or profiles of genes in a sample from a subject of interest compared to the expression levels or profiles of the same genes in one or more reference or control samples is indicative of a specific inflammatory phenotype. For example, suitable reference or control expression levels or profiles may be determined in one or more, typically a population, of individuals without an inflammatory phenotype of interest or known not to suffer from COPD. Alternatively, suitable reference or control expression levels or profiles may be determined in one or more, typically a population, of individuals known to have COPD and in which the COPD inflammatory phenotype is known or not known. In subjects to which methods disclosed herein are applied, a comparison of the expression levels or profiles with those obtained from the appropriate reference or control may determine diagnosis.

Reliable diagnoses of COPD inflammatory phenotypes, such as are possible utilising methods of the present disclosure, facilitate decision making with respect to the most appropriate intervention or treatment regime for individual subjects. The treatment regime may be tailored not only to the specific inflammatory phenotype suffered by the subject but also to the subject themselves based on one or more other factors such as the severity of the symptoms, the subjects lifestyle, age, weight, general health etc. For example, this may comprise introducing a new treatment regime or modifying an existing regime employed by the subject. The modification of a regime may be modification with respect to any one or more of a variety of factors, such as the nature of any anti-COPD medication, the dosage thereof, the timing of administration and/or any supplementary management strategies. Such decision making with respect to treatment regimes will vary from case to case and the determination of the most appropriate strategy is well within the expertise and experience of those skilled in the art.

A treatment regime for the treatment of COPD in a subject in accordance with the present disclosure may involve administration of any of the medications commonly utilised in the treatment of the disease such as bronchodilators and corticosteroids. The treatment regime may comprise the administration of a number of drugs simultaneously, sequentially, or in combination with each other or with non-drug treatments. The type of drug(s) administered, dosage, and the frequency of administration can be determined by medical physicians in accordance with accepted medical principles, and will depend on factors such as the severity of the disease, the age and weight of the subject, the medical history of the subject, other medication being taken by the subject, existing ailments and any other health related factors normally considered when determining treatments for obstructive airways disease.

The present disclosure also provides kits suitable for use in accordance with the methods of the disclosure. Such kits include for example diagnostic kits for assaying biological samples, comprising an agent(s) for detecting expression levels discriminatory biomarkers disclosed herein (such as nucleic acid molecules or proteins), and reagents useful for facilitating the determination of expression by the agent(s). The agent(s) may be any suitable detecting molecule. Kits according to the present disclosure may also include other components required to conduct the methods of the present invention, such as buffers and/or diluents. The kits typically include containers for housing the various components and instructions for using the kit components in the methods of the present disclosure.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

The following examples are illustrative of the disclosure and should not be construed as limiting in any way the general nature of the disclosure of the description throughout this specification.

### General Methods

### Study Design and Population

In a cross-sectional study, the inventors recruited non-smoking participants (n=164) with stable physician-diagnosed COPD from the respiratory ambulatory care clinics at John Hunter Hospital (Newcastle, Australia), the clinical research databases of the Priority Research Centre for Healthy Lungs at the University of Newcastle and the Hunter Medical Research Institute (Newcastle, Australia). COPD diagnosis was confirmed by incompletely reversible airflow limitation (post-bronchodilator forced expiratory volume in one second (FEV₁) <80% predicted and FEV₁ to forced vital capacity (FVC) ratio of <0.70). Participation was delayed if the participant had been treated with antibiotics or oral corticosteroids for an acute exacerbation of COPD within the previous 4 weeks. Exclusion criteria included current smoking and unstable COPD. The Hunter New England Local Health District and the University of Newcastle Human Ethics Research Committees approved this study and all participants gave written informed consent.

### Clinical Assessments

Participants attended a single visit to assess demographics, smoking status, exacerbation history in the preceding year, medical history, medication use, comorbidities (Charlson Comorbidity Index, CCI) (Charlson et al., 1987, J Chronic Dis 40:373-383) and health related quality of life (Saint George Respiratory Questionnaire, SGRQ) (Jones et al., 1992, Am Rev Respir Dis 145:1321-1327). A 6-minute walk test was performed and the BODE (Body mass index, airflow Obstruction, Dyspnoea and Exercise capacity) index was also calculated (Cell et al., 2004, N Engl J Med 350:1005-1012). Pre and post bronchodilator spirometry and sputum induction were performed (Gibson et al., 1998, Am J Respir Crit Care Med 158:36-41). Peripheral venous blood was collected and serum high-sensitivity C-reactive protein (hs-CRP) and interleukin 6 (IL-6) were measured using enzyme-linked immunosorbent assay. A subgroup of participants (n=22) were assessed approximately 1 month later, whereby a second sputum induction was carried out for assessment of reproducibility.

### Sputum Induction and Inflammatory Cell Analysis

Airflow limitation was assessed using spirometry (Medgraphics, CPFS/D™ usb Spirometer, BreezeSuite v7.1, Saint Paul, USA). Sputum induction with hypertonic saline (4.5%) was performed in participants whose FEV₁ was ≥1L according to Gibson et al, 1998, Am J Respir Crit Care Med 158:36-41. In those with FEV₁ <1L, 0.9% saline was used. Sputum was processed within thirty minutes of collection. For inflammatory cell counts, selected sputum was dispersed using dithiothreitol, and total cell count viability was performed. Cytospins were prepared, stained (May-Grunwald—Giemsa) and a differential cell count obtained from 400 non-squamous cells. For gene expression, Buffer RLT (Qiagen, Hilden, Germany) was immediately added to 100 μL of selected sputum and stored at −80° C. until RNA extraction.

### Phenotype Characteristics

Eosinophilic COPD was defined as sputum eosinophil count of 3% or more (Pavord et al., 1999, Lancet 353:2213-2214). The inventors defined neutrophilic COPD as sputum neutrophil count of >61%. Participants were deemed to have paucigranulocytic COPD if their sputum neutrophil and eosinophil counts were less than 61% and 3%, respectively. Mixed granulocytic COPD was defined as sputum neutrophil counts >61% and sputum eosinophils of ≥3%.

### Gene Expression Analysis

Sputum gene expression of CLC, CPA3, DNASE1L3, IL1B, ALPL, and CXCR2 was performed as previously described (Baines et al., 2014, J Allergy Clin Immunol 133:997-1007; Baines et al., 2011, J Allergy Clin Immunol 127:153-160). Briefly, sputum RNA was extracted using the Qiagen RNeasy Mini Kit, quantified, reverse-transcribed to cDNA and used to detect gene expression using standard Taqman real-time quantitative polymerase chain reaction (qPCR) methods (Applied Biosystems, Foster City, USA). Statistical analysis of diagnostic ability was performed on the change in cycle threshold (ΔCt) between the target gene and housekeeping β-actin. For relative gene expression levels, data were log transformed (2^{-Δct}) and scaled.

### Statistical Analysis

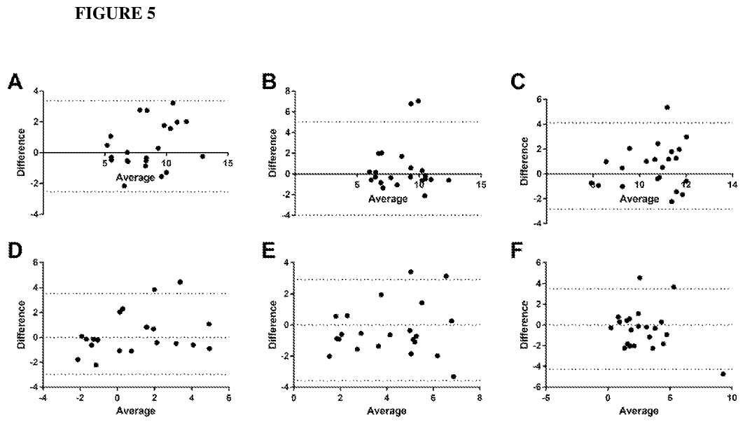

Data were analyzed using Stata 13 (Stata Corporation, College Station, Tex., USA) and were reported as mean (SD) or median (quartile 1, quartile 3) depending on the distribution. Comparisons between two independent groups were performed using Student's t-test or Wilcoxon Rank Sum test. Fisher's exact test was used to test categorical data. Comparisons between multiple groups were assessed using one-way ANOVA with Bonferoni correction. Associations were assessed using Pearson or Spearman correlation. Biomarker potential was assessed using multiple logistic regression, receiver operating characteristic curves (ROCs) and area under the curve (AUC). Significance was accepted when p<0.05. Reproducibility was assessed using intra-class correlation (ICC, MedCalc software), and Bland-Altman plots (GraphPad Prism 7). Significance was accepted when p<0.05 (FIG. 5).

Logistic regression was used to calculate the predicted value of an individual having the particular COPD inflammatory phenotype based on the expression (cycle threshold (Ct)) of each target gene compared to β-actin (ΔCt) using multiple logistic regression for the combination of 6 genes to generate one set of predicted values, as described in Baines et al., 2014, J Allergy Clin Immunol 133:997-1007. This was generated in Stata 13 statistical package. The resulting output was a set of predicted value cut points which had ranging sensitivities and specificities plotted on a receiver operating characteristic (ROC) curve, which was used to calculate the area under the curve (AUC) for the level of accuracy to correctly classify people, as well as a p value of significance for the regression itself.

### Example 1

### Clinical Characteristics and COPD Inflammatory Phenotypes

The demographics and clinical characteristics of the study population are presented in Table 1. Briefly, participants had a median (IQR) age of 70 (64, 75) years and moderate airflow limitation with a mean (SD) post-bronchodilator FEV₁ % predicted of 57 (16.5%) (Table 1). There were 91 (55.5%) males and 73 (44.5%) females, and 124 (75.6%) were ex-smokers with a median (IQR) pack years of 33 (0.4, 63). Almost half of the participants (78, 47.6%) were frequent exacerbators with a prior year exacerbation history of 2 or more. 145 (88.4%) participants were taking inhaled corticosteroids (ICS) with a median daily dose of 800 (400, 1600) μg beclomethasone equivalents/day.

TABLE 1
Clinical characteristics of the cohort

| Characteristics | Value | |
|---|---|---|
| Number | 164 | |
| Age (years), mean (SD) | 69 | (8) |
| Gender, Male n (%) | 91 | (56) |
| BMI (kg/m²), median (Q1, Q3) | 28.7 | (24.5, 33) |
| Ex-smoker, n (%) | 124 | (76) |
| Pack years, median (Q1, Q3) | 33.0 | (0.4, 63.0) |
| Post β2 FEV₁ % predicted, mean (SD) | 57 | (16) |
| Post β2 FVC % predicted, mean (SD) | 75 | (18) |
| Post β2 FEV₁/FVC, mean (SD) | 54 | (13) |
| GOLD grades, n(%) | | |
| 1 | 11 | (7) |
| 2 | 101 | (62) |
| 3 | 38 | (23) |
| 4 | 14 | (8) |
| BDR, n (%) | 70 | (43) |
| ICS use, n (%) | 145 | (88) |
| ICS dose, BDP equivalent mcg/day, median | 800 | (400, 1600) |
| (Q1, Q3) | | |
| Frequent exacerbators, n (%) | 78 | (48) |
| CCI, mean (SD) | 4.0 | (1.3) |
| SGRQ total (n = 129), mean (SD) | 47 | (18) |
| BODE (n = 110), median (Q1, Q3) | 3 | (1, 4) |
| Sputum total cell count (×10⁶/mL), median | 4.7 | (2.8, 8.7) |
| (Q1, Q3) | | |
| Sputum neutrophil %, median (Q1, Q3 | 57.4 | (36.3, 73) |
| Sputum eosinophil %, median (Q1, Q3 | 1.8 | (0.8, 4) |
| Serum CRP (n = 159), median (Q1, Q3 | 4.1 | (1.7, 8.5) |

Abbreviations: BMI, Body mass index; BODE, body mass index, airflow obstruction, dyspnoea and exercise capacity; BDR, bronchodilator responsiveness; CCI, Charlson Comorbidity Index; CRP, C-reactive protein; GOLD, Global Initiative for Chronic Obstructive Lung Disease; ICS, inhaled corticosteroid; SGRQ, Saint George Respiratory Questionnaire.

Eosinophilic inflammation alone was identified in 36 (22%) participants, neutrophilic inflammation alone was identified in 55 (34%) participants, and both types of inflammation were demonstrated in 20 (12%) participants. The remainder (n=53, 32%) had a paucigranulocytic pattern. Comparison of demographic features, clinical characteristics and sputum cell counts between patients with eosinophilic and non-eosinophilic (NE) COPD, as well as between those with neutrophilic and non-neutrophilic (NN) COPD is summarized in Table 2.

All clinical characteristics were similar between patients with E-COPD and those with no evidence of sputum eosinophilia, except for a slightly higher CCI score in the latter (Table 2). Neutrophilic inflammation was associated with lower lung function, female gender and lower BMI (Table 2).

TABLE 2
Clinical characteristics and inflammatory cells of COPD participants with and without eosinophilic or neutrophilic inflammation.

| Characteristics | E-COPD | | NE-COPD | | P value | N-COPD | | NN-COPD | | P value |
|---|---|---|---|---|---|---|---|---|---|---|
| Number (%) | 56 | (34) | 108 | (66) | N/A | 75 | (46) | 89 | (54) | N/A |
| Age, mean (SD) | 70 | (8) | 68 | (9) | 0.053 | 69 | (7) | 69 | (9) | 0.708 |
| Gender, Male, n (%) | 32 | (57) | 59 | (55) | 0.869 | 34 | (45) | 57 | (64) | 0.016 |
| BMI (kg/m²), median (Q1, Q3) | 28.8 | (24.5, 32.5) | 28.7 | (24.6, 33.4) | 0.959 | 27.6 | (23.6, 31.8) | 30.1 | (25.9, 34.2) | 0.014 |
| Ex-smoker, n (%) | 40 | (71.4) | 84 | (77.8) | 0.444 | 57 | (76) | 67 | (75) | 1.000 |
| Pack years, median (Q1, Q3) | 33.5 | (0, 53.4) | 30.6 | (1, 69.6) | 0.451 | 23.0 | (0.3, 56.3) | 37.0 | (1.0, 70.0) | 0.148 |
| Post β2 FEV₁% predicted, mean(SD) | 57 | (16) | 57 | (17) | 0.979 | 53 | (17) | 60 | (16) | 0.008 |
| Post β2 FVC % predicted, mean(SD) | 75 | (19) | 76 | (17) | 0.746 | 71 | (18) | 79 | (17) | 0.002 |
| Post β2 FEV1/FVC, mean(SD) | 54 | (13) | 54 | (13) | 0.888 | 53 | (12) | 55 | (14) | 0.303 |
| BDR, n (%) | 21 | (38) | 49 | (45) | 0.406 | 30 | (40) | 40 | (45) | 0.524 |
| ICS use, n (%) | 49 | (88) | 96 | (89) | 0.801 | 68 | (91) | 77 | (87) | 0.408 |
| ICS dose, BDP equivalent mcg/day | 500 | (250-1000) | 800 | (500-2000) | 0.109 | 500 | (500-1500) | 800 | (250-2000) | 0.747 |
| Frequent exacerbations, n (%) | 25 | (45) | 53 | (49) | 0.624 | 34 | (45) | 44 | (49) | 0.600 |
| CCI, mean(SD) | 3.7 | (1.4) | 4.1 | (1.2) | 0.023 | 4.0 | (1.2) | 4.0 | (1.4) | 0.776 |
| SGRQ total, mean(SD) | 45 | (20) | 48 | (17) | 0.388 | 45 | (17) | 48 | (19) | 0.255 |
| | (n = 41) | | (n = 88) | | | (n = 54) | | (n = 75) | | |
| BODE, median (Q1, Q3) | 2.5 | (1.0, 4.0) | 3.0 | (2.0-5.0) | 0.308 | 3.0 | (2.0-5.0) | 2.5 | (1.0-4.0) | 0.262 |
| | (n = 32) | | (n = 78) | | | (n = 46) | | (n = 64) | | |
| Sputum total cell count, ×10⁶/mL, median | 4.5 | (2.8, 8.9) | 4.7 | (2.8, 8.7) | 0.927 | 7.9 | (4.7, 14.7) | 3.2 | (2.2, 4.8) | <0.001 |
| (Q1, Q3) | | | | | | | | | | |
| Sputum neutrophil %, median (Q1, Q3) | 48.8 | (32.8, 70.4) | 61.9 | (38.8, 76.3) | 0.021 | 74.5 | (67.5, 85.5) | 40.0 | (27.5, 48.5) | <0.001 |
| Sputum eosinophil %, median (Q1, Q3) | 6.8 | (3.9, 21.8) | 1 | (0.6, 1.8) | <0.001 | 1.5 | (0.8, 3.0) | 2.0 | (0.8-7.0) | 0.047 |

Data presented as n (%), mean (SD) or median (quartile 1-3).
Abbreviations:
E-COPD: Eosinophilic - Chronic Obstructive Pulmonary Disease;
NE-COPD: Non-Eosinophilic-COPD;
N-COPD: Neutrophilic-COPD;
NN-COPD: Non-Neutrophilic COPD;
BMI: body mass index;
FEV₁: Forced expiratory volume in 1 sec;
FVC: forced vital capacity;
BDR: Bronchodilator responsiveness;
ICS: inhaled corticosteroid;
CCI: Charlson Comorbidity Index;
SGRQ: Saint George Respiratory Questionnaire;
BODE: Body mass index, airflow Obstruction, Dyspnoea and Exercise capacity

### Example 2

### Sputum 6-Gene Expression Signature and Inflammatory Phenotypes in COPD

CLC, CPA3 and DNASE1L3 expression levels were significantly higher in patients with eosinophilic COPD (E-COPD) compared to non-eosinophilic COPD (NE-COPD) (Table 3). Higher expression levels of IL1B was observed in NE-COPD, while there was no difference in ALPL or CXCR2 expression between the two groups. IL1B, ALPL and CXCR2 expression levels were significantly higher in patients with N-COPD compared to NN-COPD (Table 3), whereas there was no difference in CLC, CPA3 and DNASE1L3 expression between these groups.

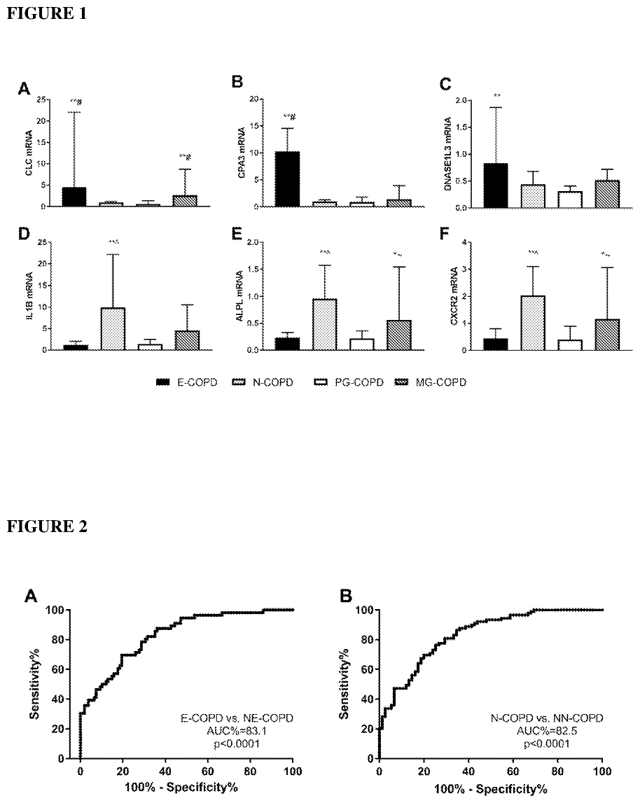

Furthermore, when dividing the COPD participants into 4 inflammatory phenotypes (eosinophilic, E-COPD; neutrophilic, N-COPD; paucigranulocytic, PG-COPD; and mixed granulocytic, MG-COPD), CLC expression was higher in E-COPD and MG-COPD compared with N-COPD and PG-COPD. Sputum gene expression of CPA3 was higher in E-COPD compared with N-COPD, PG-COPD and MG-COPD, whereas DNASE1L3 was higher in E-COPD compared with PG-COPD. Sputum gene expression of IL1B, ALPL and CXCR2 were higher in N-COPD and MG-COPD compared with E-COPD and PG-COPD (FIG. 1).

TABLE 3
Sputum gene expression levels of COPD participants with
and without eosinophilic or neutrophilic inflammation

| Marker | E-COPD | | NE-COPD | | P value | N-COPD | | NN-COPD | | P value |
|---|---|---|---|---|---|---|---|---|---|---|
| n | 56 | | 108 | | | 75 | | 89 | | |
| CLC | 2.9 | (1.3, 22.6) | 0.8 | (0.3, 1-9) | <0.001 | 1.2 | (0.5, 3.4) | 1.2 | (0.4, 4.2) | 0.965 |
| mRNA | | | | | | | | | | |
| CPA3 | 4.8 | (1.0, 12.2) | 0.9 | (0.4, 1.9) | <0.001 | 1.1 | (0.6, 1.9) | 1.8 | (0.5, 6.8) | 0.051 |
| mRNA | | | | | | | | | | |
| DNASE1L3 | 0.7 | (0.3, 1.7) | 0.4 | (0.2, 0.7) | 0.003 | 0.4 | (0.2, 1.1) | 0.4 | (0.2, 0.9) | 0.568 |
| mRNA | | | | | | | | | | |
| IL1B | 1.9 | (0.7, 6.6) | 3.6 | (0.9, 14.6) | 0.047 | 6.5 | (2.1, 24.2) | 1.3 | (0.5, 3.7) | <0.001 |
| mRNA | | | | | | | | | | |
| ALPL | 0.3 | (0.1, 0.6) | 0.5 | (0.2, 1.1) | 0.082 | 0.8 | (0.4, 1.9) | 0.2 | (0.1, 0.4) | <0.001 |
| mRNA | | | | | | | | | | |
| CXCR2 | 0.6 | (0.3, 1.4) | 1.0 | (0.3, 2.3) | 0.113 | 1.9 | (0.7, 3.7) | 0.4 | (0.2, 1.2) | <0.001 |
| mRNA | | | | | | | | | | |

Data expressed as 2^{-ΔCt} compared with the housekeeping gene β-actin (median (Q1, Q3)).
Abbreviations:
E-COPD: eosinophilic COPD;
NE-COPD: non-eosinophilic COPD.

### Example 3

### Diagnostic Performance of Sputum 6-Gene Expression Signature in Predicting Airway Inflammatory Phenotypes in COPD

The diagnostic performance of the 6-gene signature, a composite of gene expression results for CLC, CPA3, DNASE1L3, IL1B, ALPL and CXCR2, was evaluated for predicting the different inflammatory phenotypes of COPD (Table 4). Firstly, the expression levels of the 6-genes in combination was able to identify participants with COPD that had eosinophilic inflammation compared to those without eosinophilic inflammation (FIG. 2A, E-COPD vs. NE-COPD; AUC=83.1%; 95% CI (76.8-89.5%); p<0.0001), as well as those participants with COPD that had neutrophilic inflammation compared with those without neutrophilic inflammation (FIG. 2B, N-COPD vs. NN-COPD; AUC=83.4; 95% CI (77.3-89.4%); p<0.0001).

At an optimal predicted value cut point of 0.288 (sensitivity=78.6%, specificity=71.3% and positive likelihood ratio=2.74), the sputum 6-gene signature can help correctly identify E-COPD from NE-COPD in 74 of 100 cases. At an optimal predicted value cut point of 0.522 (sensitivity=73.3%, specificity=77.5% and positive likelihood ratio=3.3), the sputum 6-gene signature can help correctly identify N-COPD from NN-COPD in 76 of 100 cases.

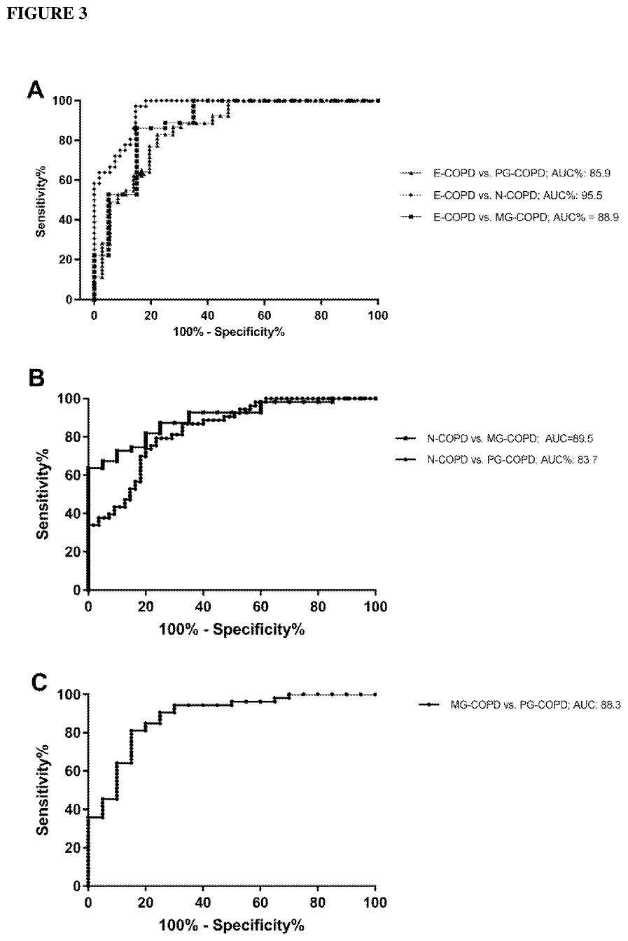

Furthermore, when splitting the participants into 4 inflammatory phenotypes, the 6 gene expression signature could discriminate E-COPD from PG-COPD (AUC%=85.9; 95% CI=77.7-94.1; p<0.0001), N-COPD (AUC%=95.5; 95% CI=91.9-99.1; p<0.0001), and MG-COPD (AUC%=88.9; 95% CI=78.9-98.9; p<0.0001) (FIG. 3A). The 6-gene expression signature also distinguished N-COPD from PG-COPD (AUC%=83.7; 95% CI=76.3-91.0; p<0.0001) and MG-COPD (AUC%=89.5; 95% CI=82.3-96.6; p<0.0001) (FIG. 3B), as well as MG-COPD from PG-COPD (AUC%=88.3; 95% CI=79.2-97.4; p<0.0001) (FIG. 3C).

The optimal predicted cut points for the 6-gene expression signature to distinguish E-COPD from N-COPD, PG-COPD and MG-COPD were 0.312 (sensitivity=97.2%, specificity=85.5% and positive likelihood ratio=6.7, correctly classified 90%), 0.482 (sensitivity=72.2%, specificity=86.8% and positive likelihood ratio=5.5, correctly classified 81%), and 0.674 (sensitivity=86.1%, specificity=85.0% and positive likelihood ratio=5.7, correctly classified 86%), respectively. The optimal predicted cut points for the 6-gene expression signature to distinguish N-COPD from PG-COPD and MG-COPD were 0.543 (sensitivity=76.4%, specificity=79.3% and positive likelihood ratio=3.7, correctly classified 78%), and 0.674 (sensitivity=87.3%, specificity=75.0% and positive likelihood ratio=3.5, correctly classified 84%), respectively. The optimal predicted cut point for the 6 gene expression signature to distinguish MG-COPD from PG-COPD was 0.653 (sensitivity=84.9%, specificity=80.0%, and positive likelihood ratio=4.2, correctly classified 84%).

To assess reproducibility, sputum gene expression of the 6 biomarkers was measured in 22 subjects (n=9 E-COPD, n=9 N-COPD, n=4 PG-COPD) on 2 occasions, a mean (SD) of 37 (20) days apart. The bias of measurement was small with equal scatter for all genes (data not shown). ICC coefficients were excellent for CLC (0.78) and IL1B (0.76), good for ALPL (0.65) and CXCR2 (0.60), fair for CPA3 (0.45), and poor for DNASE1L3 (0.33).

### Example 4

### Correlations of Gene Expression Markers with Clinical Outcomes

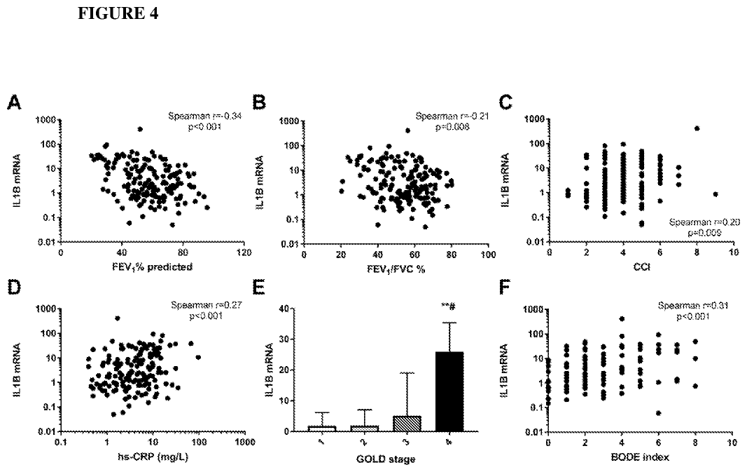

Elevated gene expression levels of IL1B had weak but significant associations with post-bronchodilator FEV₁% predicted (r=−0.34; p<0.0001; FIG. 4A), FEV₁/FVC ratio (r=−0.21; p=0.008; FIG. 4B) and CCI scores (r=0.21; p=0.008; FIG. 4C). Elevated expression of ALPL and CXCR2 genes also correlated with poor lung function (FEV₁ % predicted r=−0.32; p<0.001; and r=−0.24; p=0.002, respectively). Elevated gene expression of IL1B (FIG. 4D; r=0.27; p<0.001) and ALPL (r=0.22; p=0.006) were associated with systemic inflammation (elevated hs-CRP). Gene expression of IL1B (FIG. 4E; p<0.001), ALPL (p<0.001) and CXCR2 (p=0.017) was significantly higher in participants in GOLD grade 4 especially when compared with those in GOLD grades 1 and 2. Neutrophil related signatures were associated with BODE index (IL1B: FIG. 4F; r=0.31; p<0.001, ALPL: r=0.30; p=0.002, CXCR2: r=0.20; p=0.041). CLC, CPA3 and DNASE1L3 did not show any correlations with above mentioned clinical outcomes.

TABLE 4
Diagnostic value of CLA, CPA3, DNASE1L3, IL1β, ALPL, and CXCR2 in combination for inflammatory phenotype of COPD

| | | | | | Minimal False Negatives | | | Minimal False Positives | | |
|---|---|---|---|---|---|---|---|---|---|---|
| | | | | | Predicted | | | Predicted | | |
| | Logistic regression | | | Model | Value Cut | | | Value Cut | | |
| Comparison | Coefficient | Constant | AUC % | p value | Point | Sensitivity | Specificity | Point | Sensitivity | Specificity |
| E-COPD vs. NE- | −0.5695114 | 3.757392 | 83.1 | <0.0001 | ≥0.256 | 82.1% | 68.5% | ≥0.3339 | 71.4% | 74.1% |
| COPD | | | | | | | | | | |
| CLC | | | | | | | | | | |
| CPA3 | −0.2310326 | | | | | | | | | |
| DNASE1L3 | 0.1606380 | | | | | | | | | |
| IL1B | 0.1762747 | | | | | | | | | |
| ALPL | 0.3150170 | | | | | | | | | |
| CXCR2 | −0.1765517 | | | | | | | | | |
| N-COPD vs. NN- | | −0.455391 | 83.4 | <0.0001 | ≥0.417 | 76.0% | 73.1% | ≥0.534 | 70.7% | 80.9% |
| COPD | | | | | | | | | | |
| CLC | 0.0210132 | | | | | | | | | |
| CPA3 | 0.1548456 | | | | | | | | | |
| DNASE1L3 | 0.1664075 | | | | | | | | | |
| IL1B | −0.2003341 | | | | | | | | | |
| ALPL | −0.4577762 | | | | | | | | | |
| CXCR2 | −0.2252791 | | | | | | | | | |
| E-COPD vs. PG- | | 7.867614 | 85.9 | <0.0001 | ≥0.376 | 80.6% | 77.4% | ≥0.492 | 69.4% | 88.7% |
| COPD | | | | | | | | | | |
| CLC | −0.2909536 | | | | | | | | | |
| CPA3 | −0.3370669 | | | | | | | | | |
| DNASE1L3 | −0.3850054 | | | | | | | | | |
| IL1B | 0.3057671 | | | | | | | | | |
| ALPL | 0.4355524 | | | | | | | | | |
| CXCR2 | −0.3526798 | | | | | | | | | |
| E-COPD vs. N- | | 6.107679 | 95.5 | <0.0001 | ≥0.312 | 97.2% | 85.5% | ≥0.584 | 75.0% | 90.9% |
| COPD | | | | | | | | | | |
| CLC | −1.084175 | | | | | | | | | |
| CPA3 | −0.4605654 | | | | | | | | | |
| DNASE1L3 | 0.1305373 | | | | | | | | | |
| IL1B | 0.4013217 | | | | | | | | | |
| ALPL | 1.161539 | | | | | | | | | |
| CXCR2 | 0.0934771 | | | | | | | | | |
| E-COPD vs. | | 3.873287 | 88.9 | 0.0001 | ≥0.510 | 88.9% | 75.0% | ≥0.674 | 86.1% | 85.0% |
| MG-COPD | | | | | | | | | | |
| CLC | −0.4392087 | | | | | | | | | |
| CPA3 | 0.5989352 | | | | | | | | | |
| DNASE1L3 | 0.521669 | | | | | | | | | |
| IL1B | −0.0188731 | | | | | | | | | |
| ALPL | 0.8532774 | | | | | | | | | |
| CXCR2 | −0.0528469 | | | | | | | | | |
| N-COPD vs. PG- | | 1.164648 | 83.7 | <0.0001 | ≥0.502 | 80.0% | 73.6% | ≥0.543 | 76.4% | 79.3% |
| COPD | | | | | | | | | | |
| CLC | 0.1245468 | | | | | | | | | |
| CPA3 | −0.0616497 | | | | | | | | | |
| DNASE1L3 | 0.1322279 | | | | | | | | | |
| IL1B | −0.2454009 | | | | | | | | | |
| ALPL | −0.3834751 | | | | | | | | | |
| CXCR2 | −0.3498634 | | | | | | | | | |
| N-COPD vs. | | 0.6367382 | 89.5 | <0.0001 | ≥0.674 | 87.3% | 75.0% | ≥0.843 | 72.7% | 90.0% |
| MG-COPD | | | | | | | | | | |
| CLC | 1.112398 | | | | | | | | | |
| CPA3 | 0.1087039 | | | | | | | | | |
| DNASE1L3 | −0.7844116 | | | | | | | | | |
| IL1B | −0.2713489 | | | | | | | | | |
| ALPL | −0.0970896 | | | | | | | | | |
| CXCR2 | −0.108696 | | | | | | | | | |
| MG-COPD vs. | | −5.231813 | 88.3 | <0.0001 | ≥0.583 | 90.6% | 75.0% | ≥0.744 | 81.1% | 85.0% |
| PG-COPD | | | | | | | | | | |
| CLC | 0.9125754 | | | | | | | | | |
| CPA3 | −0.1599103 | | | | | | | | | |
| DNASE1L3 | −0.3600558 | | | | | | | | | |
| IL1B | −0.0882882 | | | | | | | | | |
| ALPL | 0.5221864 | | | | | | | | | |
| CXCR2 | 0.3208307 | | | | | | | | | |

Minimal false negatives correspond to the point of the ROC curve with the highest sensitivity (true positive rate, useful for ruling disease out) whereas minimal false positives correspond to the point with the highest specificity (false positive rate, useful for ruling disease in). E-COPD: eosinophilic COPD, MG-COPD mixed granulocytic COPD, NE-COPD: non-eosinophilic COPD, PG-COPD: paucigranulocytic COPD, N-COPD: neutrophilic COPD, NN-COPD: non-neutrophilic COPD, AUC: area under the curve.

1. A method for determining an inflammatory phenotype of chronic obstructive pulmonary disease (COPD) in a subject with COPD, the method comprising:
determining the level of expression of one or more genes in a biological sample from the subject, wherein the one or more genes are selected from CLC, CPA3, DNASE1L3, IL1B, ALPL and CXCR2;
wherein the level of expression of the one or more genes is indicative of the COPD inflammatory phenotype of the subject.

2. The method according to claim 1, wherein the correlation between expression of the one or more genes and a COPD inflammatory phenotype is determined by statistical analysis of the mRNA or protein expression levels.

3. The method according to claim 2, wherein the statistical analysis comprises logistic regression analysis.

4. The method according to claim 1, wherein the level of expression of the one or more genes is compared to the level of expression of the same gene(s) in one or more reference samples.

5. The method according to claim 4, wherein the one or more reference samples are from one or more individuals known to have COPD.

6. The method according to claim 1, wherein elevated expression of one or more of CLC, CPA3 and/or DNASE1L3 in the biological sample, compared to one or more reference samples from one or more individuals known to have COPD, is indicative of eosinophilic COPD.

7. The method according to claim 6, wherein the one or more reference samples are from one or more individuals known not to have eosinophilic COPD.

8. The method according to claim 1, wherein elevated expression of one or more of IL1B, ALPL and/or CXCR2 in the biological sample, compared to one or more reference samples from one or more individuals known to have COPD, is indicative of neutrophilic COPD.

9. The method according to claim 8, wherein the one or more reference samples are from one or more individuals known not to have neutrophilic COPD.

10. The method according to claim 1, wherein elevated expression of IL1B in the biological sample, compared to one or more reference samples from one or more individuals known to have COPD, is indicative of non-eosinophilic COPD.

11. The method according to claim 10, wherein the one or more reference samples are from one or more individuals known to have eosinophilic COPD.

12. The method according to claim 1, wherein the combined expression profile of CLC, CPA3, DNASE1L3, IL1B, ALPL and CXCR2 in the biological sample is compared to the combined expression profile of said genes in one or more reference samples from one or more individuals known to have COPD.

13. The method according to claim 12, wherein the COPD inflammatory phenotype of said one or more individuals, from which the one or more reference samples are derived, is known.

14. The method according to claim 1, wherein the biological sample is sputum.

15. The method according to claim 14, wherein the sputum is induced sputum.

16. A method for determining a COPD inflammatory phenotype in a subject with COPD, the method comprising:
determining the expression profile, in a biological sample from the subject, of the genes CLC, CPA3, DNASE1L3, IL1B, ALPL and CXCR2;
wherein the expression profile of said genes is indicative of the COPD inflammatory phenotype of the subject.

17. The method according to claim 16, wherein the correlation between the expression profile of the genes and a COPD inflammatory phenotype is determined by statistical analysis of the mRNA or protein expression levels.

18. The method according to claim 17, wherein the statistical analysis comprises logistic regression analysis.

19. The method according to claim 16, wherein multiple logistic regression analysis of the expression profile or expression levels of said genes enables the distinction between:
a) eosinophilic COPD and non-eosinophilic COPD;
b) eosinophilic COPD and neutrophilic COPD;
c) eosinophilic COPD and paucigranulocytic COPD;
d) eosinophilic COPD and mixed granulocytic COPD;
e) neutrophilic COPD and non-neutrophilic COPD;
f) neutrophilic COPD and paucigranulocytic COPD;
g) neutrophilic COPD and mixed granulocytic COPD;
h) paucigranulocytic COPD and non-paucigranulocytic COPD; or
i) paucigranulocytic COPD and mixed granulocytic COPD.

20. The method according to claim 16, wherein the level of expression of the one or more genes is compared to the level of expression of the same gene(s) in one or more reference samples.

21. The method according to claim 20, wherein the one or more reference samples are from one or more individuals known to have COPD.

22. The method according to claim 16, wherein the biological sample is sputum.

23. The method according to claim 22, wherein the sputum is induced sputum.

24. A method for selecting a subject for treatment of an inflammatory phenotype of COPD, comprising:
i) executing the step of determining the level of expression of one or more genes in a biological sample from a subject, wherein the one or more genes are selected from CLC, CPA3, DNASE1L3, IL1B, ALPL and CXCR2;
ii) determining the COPD inflammatory phenotype based on the determination in i); and
iii) selecting a subject for treatment for said COPD inflammatory phenotype determined in ii).

25. The method for selecting a subject for treatment of an inflammatory phenotype of COPD according to claim 24, wherein:
step i) comprises executing the step of determining the expression profile, in a biological sample from a subject, of the genes CLC, CPA3, DNASE1L3, IL1B, ALPL and CXCR2.

26. The method for determining a treatment regime for a subject suffering from COPD, the method comprising determining the COPD inflammatory phenotype in the subject in accordance with claim 1 and selecting an appropriate treatment regime for the subject on the basis of the determination.

27. A method for determining a treatment regime for a subject suffering from COPD, the method comprising determining the COPD inflammatory phenotype in the subject in accordance with claim 16 and selecting an appropriate treatment regime for the subject on the basis of the determination.


SEQUENCE LISTING (12 sequences)

SEQ ID NO: 1 - DNA, 429 bp, Homo sapiens
atgtccctgctacccgtgccatacacagaggctgcctctttgtctactggttctactgtgacaatcaaagggcgaccacttgcctgtttcttgaatgaaccatatctgcaggtggatttccacactgagatgaaggaggaatcagacattgtcttccatttccaagtgtgctttggtcgtcgtgtggtcatgaacagccgtgagtatggggcctggaagcagcaggtggaatccaagaatatgccctttcaggatggccaagaatttgaactgagcatctcagtgctgccagataagtaccaggtaatggtcaatggccaatcctcttacacctttgaccatagaatcaagcctgaggctgtgaagatggtgcaagtgtggagagatatctccctgaccaaatttaatgtcagctatttaaagagataa

SEQ ID NO: 2 - protein, 142 aa, Homo sapiens
MSLLPVPYTEAASLSTGSTVTIKGRPLACFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLKR

SEQ ID NO: 3 - DNA, 1254 bp, Homo sapiens
atgaggctcatcctgcctgtgggtttgattgctaccactcttgcaattgctcctgtccgctttgacagggagaaggtgttccgcgtgaagccccaggatgaaaaacaagcagacatcataaaggacttggccaaaaccaatgagcttgacttctggtatccaggtgccacccaccacgtagctgctaatatgatggtggatttccgagttagtgagaaggaatcccaagccatccagtctgccttggatcaaaataaaatgcactatgaaatcttgattcatgatctacaagaagagattgagaaacagtttgatgttaaagaagatatcccaggcaggcacagctacgcaaaatacaataattgggaaaagattgtggcttggactgaaaagatgatggataagtatcctgaaatggtctctcgtattaaaattggatctactgttgaagataatccactatatgttctgaagattggggaaaagaatgaaagaagaaaggctatttttacggattgtggcattcacgcacgagaatgggtctccccagcattctgccagtggtttgtctatcaggcaaccaaaacttatgggagaaacaaaattatgaccaaactcttggaccgaatgaatttttacattcttcctgtgttcaatgttgatggatatatttggtcatggacaaagaaccgcatgtggagaaaaaatcgttccaagaaccaaaactccaaatgcatcggcactgacctcaacaggaattttaatgcttcatggaactccattcctaacaccaatgacccatgtgcagataactatcggggctctgcaccagagtccgagaaagagacgaaagctgtcactaatttcattagaagccacctgaatgaaatcaaggtttacatcaccttccattcctactcccagatgctattgtttccctatggatatacatcaaaactgccacctaaccatgaggacttggccaaagttgcaaagattggcactgatgttctatcaactcgatatgaaacccgctacatctatggcccaatagaatcaacaatttacccgatatcaggttcttctttagactgggcttatgacctgggcatcaaacacacatttgcctttgagctccgagataaaggcaaatttggttttctccttccagaatcccggataaagccaacgtgcagagagaccatgctagctgtcaaatttattgccaagtatatcctcaagcatacttcctaa

SEQ ID NO: 4 - protein, 417 aa, Homo sapiens
MRLILPVGLIATTLAIAPVRFDREKVFRVKPQDEKQADIIKDLAKTNELDFWYPGATHHVAANMMVDFRVSEKESQAIQSALDQNKMHYEILIHDLQEEIEKQFDVKEDIPGRHSYAKYNNWEKIVAWTEKMMDKYPEMVSRIKIGSTVEDNPLYVLKIGEKNERRKAIFTDCGIHAREWVSPAFCQWFVYQATKTYGRNKIMTKLLDRMNFYILPVFNVDGYIWSWTKNRMWRKNRSKNQNSKCIGTDLNRNFNASWNSIPNTNDPCADNYRGSAPESEKETKAVTNFIRSHLNEIKVYITFHSYSQMLLFPYGYTSKLPPNHEDLAKVAKIGTDVLSTRYETRYIYGPIESTIYPISGSSLDWAYDLGIKHTFAFELRDKGKFGFLLPESRIKPTCRETMLAVKFIAKYILKHTS

SEQ ID NO: 5 - DNA, 918 bp, Homo sapiens
atgtcacgggagctggccccactgctgcttctcctcctctccatccacagcgccctggccatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacgtacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcctag

SEQ ID NO: 6 - protein, 305 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 7 - DNA, 810 bp, Homo sapiens
atggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 8 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 9 - DNA, 1575 bp, Homo sapiens
atgatttcaccattcttagtactggccattggcacctgccttactaactccttagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagcgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctgctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcggcaggcagccttgctgcaggccccctgctgctcgcgctggccctctaccccctgagcgtcctgttctga

SEQ ID NO: 10 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 11 - DNA, 1083 bp, Homo sapiens
atggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaa

SEQ ID NO: 12 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL